(19) United States
(12) Patent Application Publication
(10) Pub. No.: US 2019/0374502 A1
(43) Pub. Date: Dec. 12, 2019

(54) Cannabinoid-Containing Fatty Acid Formulations for Treating Disorders of the Nervous System

(71) Applicant: Bodhi Research & Development Inc., Toronto (CA)

(72) Inventor: Neilank K. Jha, Toronto (CA)

(21) Appl. No.: 16/484,637

(22) PCT Filed: Feb. 9, 2018
(86) PCT No.: PCT/CA2018/050153

§ 371 Date: Aug. 8, 2019

Related U.S. Application Data
(60) Provisional application No. 62/457,059, filed on Feb. 9, 2017.

(51) Int. Cl. A61K 31/352; A61K 31/05; A61P 25/00; A61K 31/202

(52) U.S. Cl. CPC A61K 31/352; A61K 31/202; A61P 25/00; A61K 31/05

(57) ABSTRACT

Pharmaceutical compositions comprising cannabidiol (CBD), tetrahydrocannabinol (THC), and select omega-3 fatty acids are provided. The CBD and THC are present in a weight ratio of CBD:THC of 10:1 to 20:1. The omega-3 fatty acids comprise eicosapentaenoic acid and/or docosahexaenoic acid. Such compositions are useful in the treatment and prevention of traumatic brain injuries, concussions, post-concussive brain injury (PCS), and complications arising from such injuries.

### FIELD OF THE INVENTION

The invention is in the field of medicinal preparations comprising a mixture of organic active ingredients, including phenolic cannabinoids and fatty acids, as well as specific therapeutic activities of these medicinal preparations in treating disorders of the nervous system.

### BACKGROUND OF THE INVENTION

An estimated 1.6-3.8 million traumatic brain injuries (TBIs) occur in the United States each year, a majority of which are concussions (Langlois, Rutland-Brown et al. 2006). The main pathophysiological mechanism underlying concussion is thought to be a neurometabolic cascade initiated by biomechanical injury starting with acute ionic flux and glutamate release, leading to mechanoporation of lipid membranes, causing aberrant downstream effects on voltage-gated and ligand-gated ion channels (Giza and Hovda 2014). This culminates in an energy crisis with an acute hypermetabolic phase, followed by a longer hypometabolic phase of 7 to 10 days. Other key sequelae of concussion include cytoskeletal damage, axonal dysfunction, altered neurotransmission, and immunoexcitotoxicity (Blaylock and Maroon 2011).

Neuroinflammation is also triggered in response to TBI (Giza and Hovda 2014). In the central nervous system (CNS), inflammation involves the recruitment of monocytes and neutrophils that secrete signaling molecules such as cytokines, which attempt to aid in the process of tissue repair (Patterson and Holahan 2012). Microglia and astrocytes of the brain are also capable of initiating their own inflammatory responses. These acute changes have been demonstrated to occur after sustaining TBI, even in the absence of symptom presentation (Broglio, Eckner et al. 2012, Shultz, MacFabe et al. 2012).

Approximately 10% of individuals who sustain a concussion develop post-concussive syndrome (PCS), where symptoms can persist for months or even years. There is an existing need for therapeutics which effectively prevent or treat the sequalae of TBI or PCS.

The two major types of cannabinoid receptors that have been identified are cannabinoid receptor 1 (CB1) and cannabinoid receptor 2 (CB2). CB1 is found in both the CNS and the periphery, with much greater expression in the CNS. CB2 is expressed predominantly by cells of the immune system, including microglia and astrocytes of the CNS, and has more recently been detected in neurons (Svizenska, Dubovy et al. 2008, Vendel and de Lange 2014). Substrates for these receptors may be classified into three categories: endocannabinoids, phytocannabinoids, and synthetic cannabinoids (Vendel and de Lange 2014). The two widely investigated endocannabinoids are anandamide (AEA) and 2-arachidonoyl glycerol (2-AG). These endocannabinoids bind and activate the same receptors as phytocannabinoids, the most well-known of which are tetrahydrocannabinol (THC) and cannabidiol (CBD) (Vendel and de Lange 2014, Woodhams, Sagar et al. 2015).

Cannabinoids have been reported to have both neuroprotective and neurotoxic effects (Same et al. Br J Pharmacol. 2011 August; 163 (7):1391-401) CBD in particular is known to show biphasic effects, an example of which is that lower doses increase wakefulness and higher doses cause a sedation effect (Chagas, Crippa et al. 2013). However, due to the complexity involved in finding a therapeutically effective dose of CBD alone for a given disorder of the nervous system, the challenge is exponentially greater when combined with THC, other cannabinoids, and terpenoids. Chronic, long-term Cannabis use in healthy people has also been associated with smaller hippocampal volumes, which implies some possible neurotoxic effect (Rocchetti, Crescini et al. 2013). Other studies involving ultra-low doses of THC in mice have shown that it leads to some long term mild cognitive deficits, even with a single dose (Same, Asaf et al. 2011).

Other studies have provided some support for neuroprotection by cannabinoids putatively via the anti-inflammatory effects of the CB2 receptor, which has been implicated in suppression of pro-inflammatory cytokine production (such as TNF-α), as well as activation of anti-inflammatory cytokines (Vendel and de Lange 2014). Evidence also exists for the participation of CB1 receptors in modulating neuroinflammation (Vendel and de Lange 2014). Modulators of the endocannabinoid system may play a role in protecting against excitotoxicity associated with NMDA receptor overactivation, mainly via CB1, and studies have indicated some mechanisms by which cannabinoids may act to oppose glutaminergic NMDA receptor activity (Fowler 2003, Sanchez-Blazquez, Rodriguez-Munoz et al. 2014, Vendel and de Lange 2014). Although CBD is also known to exhibit effects independent of CB receptors, the exact physiological mechanisms and effects, especially in combination with other molecules in a plant extract, is not well understood.

Omega-3 (docosahexaenoic acid (DHA), eicosapentaenoic acid (EPA), and α-linolenic acid) and omega-6 (linoleic acid) are essential long-chain polyunsaturated fatty acids (PUFA) that are vital for normal metabolism (Ellulu, Khaza'ai et al. 2015). DHA and EPA are the two primary omega-3 fatty acids found in humans which can be directly obtained via ingestion of fish and fish oil (Jain, Aggarwal et al. 2015), and are also produced via enzymatic conversion of α-linolenic acid, albeit at a very inefficient rate (<1%) (Bailes and Patel 2014). Palatable and stable salts of EPA and DHA have been described (WO 2007066232).

In the brain, DHA constitutes approximately 97% of total omega-3 content (Bailes and Patel 2014, Desai, Kevala et al. 2014). Studies have shown that DHA in particular is critical for brain development as well as cognitive function throughout life (Morse 2012, Bailes and Patel 2014, Barrett, McBurney et al. 2014). DHA deficient mice were shown to have decreased functional recovery, slower recovery, greater cognitive deficits, and increased neuronal death after severe TBI (Desai, Kevala et al. 2014).

### BRIEF DESCRIPTION OF THE DRAWINGS

FIG. 1 is graph illustrating Symptom Improvement Ratio for each subject in an exemplified treatment cohort. Positive ratio values indicate improvements in symptom score (symptom score reduction).

FIG. 2 is a bar graph illustrating Average Symptom Score Change across all subjects in an exemplified treatment cohort. Initial Symptom score average is 80.91. Final Symptom score average is 60.41. Error bars show standard error.

FIG. 3 is a bar graph illustrating the SCAT3 symptom score change for all subjects in an exemplified treatment cohort. The difference between initial and final scores is significant at p<0.001. Effect size as measured by Cohen's d is 1.20.

FIG. 4 is a bar graph illustrating the percentage of patients in the exemplified treatment cohort whose SCAT3 symptom score improved compared to the percentage that did not improve. 87.5% of patients reported symptom improvement on the study drug, versus 12.5% who did not report improvement.

FIG. 5 is a bar graph comparing the effect size of the exemplified treatment with the effect size of common drugs prescribed for various medical indications for prophylactic or therapeutic use.

FIG. 6 is a bar graph illustrating results from a clinical trial of 100 patients, showing % improvement in the designated symptoms.

FIG. 7 is a bar graph illustrating results from a clinical trial of 100 patients, evidencing a significant p-value (p<0.001) and treatment effect size (1.32) for the trial.

### SUMMARY OF THE INVENTION

Cannabinoid-containing therapeutic formulations are provided, comprising omega-3 fatty acids, for the treatment of disorders of the nervous system, including sequelae of concussion and traumatic brain injury (TBI), including post-concussive syndrome (PCS). In select embodiments, a CBD-rich plant extract containing a high ratio of cannabidiol (CBD) to tetrahydrocannabinol (THC) is provided, optionally in combination with various terpenoids, optionally in solution with a docosahexaenoic acid (DHA)-rich omega-3 solvent. Accordingly, in one aspect, the present invention relates to the use of cannabinoid-containing plant extracts which contain a high CBD:THC ratio, in solution with a DHA-rich solvent, in the prevention or treatment of TBI, concussion, PCS neuroinflammation or sequelae thereof.

In select embodiments, the “cannabinoid-containing plant extract” herein is an extract from a plant of the Cannabis genus, for example Cannabis sativa or Cannabis indica. A wide variety of methods may be used to prepare these plant extracts, including, but not limited to, supercritical or subcritical extraction with CO₂, extraction with hot gas, and extraction with solvents.

Also provided are methods of prophylactically or therapeutically treating a nervous system disorder, such TBI, concussion, PCS, neuroinflammation or sequelae thereof, involving the administration of an effective amount of a formulation comprising CBD and THC, for example where the weight ratio of CBD:THC is equal to or between 10:1 and 20:1, optionally wherein the CBD and THC are in solution in a DHA-rich solvent.

### DETAILED DESCRIPTION OF THE INVENTION

In one aspect, a pharmaceutical formulation is provided comprising: cannabidiol (CBD) and tetrahydrocannabinol (THC), where the weight ratio of CBD:THC is equal to or between 10:1 and 20:1 (in alternative embodiments: between 14:1 and 18:1; or approximately 16:1). Also optionally provided in the formulation are one or more omega-3 fatty acids, comprising eicosahexaenoic acid (EPA) and/or docosahexaenoic acid (DHA) or pharmaceutically acceptable salts thereof. The omega-3 fatty acid(s) may for example be provided in a concentration of 200-3000 mg/mL of EPA/DHA combined. A pharmaceutically acceptable excipient may optionally be included in the formulation, and the CBD and THC may be dissolved in the formulation.

In alternative embodiments, CBD may for example be present at 10-15 mg/ml, 12-14 mg/ml or at approximately 12.8 mg/ml. Similarly, THC may optionally be present at 0.5 to 1 mg/ml, 0.6-0.9 mg/ml or approximately 0.8 mg/ml.

In select embodiments, formulations may for example comprise: 0.8 mg/mL THC, 12.8 mg/mL CBD (or alternative amounts in the same or similar ratios, for example between 14:1 and 18:1; or approximately 16:1), for example in an approximately 1.5 mL dosage form (or 0.5-3 mL or 1-2 mL), to be taken in combination, simultaneously or sequentially, with 1-3 g DHA and/or EPA as delivery oil. Optionally, formulations may include astaxanthin (3,3′-dihydroxy-β-carotene-4,4′-dione), for example in an amount of 1-3 g in combination with 0.8 mg THC and 12.8 mg CBD (or in alternative amounts that provide the same or similar ratios of

The CBD and THC may for example be obtained from a plant extract, such as an extract of Cannabis sativa or Cannabis indica.

One or more additional cannabinoids may be provided in the formulation, such as: cannabigerol (CBG), cannabichromene (CBC), tetrahydrocannabivirin THCV), cannabidivirin (CBDV), or cannabidiolic acid (CBDA). The cannabinoid containing fraction of the formulation may be complemented with a non-cannabinoid-containing fraction, for example including one or more of: terpenes, terpenoids, sterols, triglycerides, alkanes, squalene, tocopherol, carotenoids, chlorophyll, flavonoid glycosides, or alkaloids. The non-cannabinoid-containing fraction may for example makes up between 1% and 45% by weight of the formulation, with the ratio of the cannabinoid-containing fraction to the non-cannabinoid-containing fraction being between 55:45 and 99:1.

Formulations may be used to prophylactically or therapeutically treat traumatic brain injury, concussion, post-concussive syndrome, neuroinflammation or sequelae thereof, or to formulate medicaments for these treatments. Methods are accordingly provided for prophylactically or therapeutically treating nervous system disorders of this kind, in subjects in need of such treatment, involving administering to the subject an effective amount of the formulations of the invention. Subjects amenable to treatment include mammalian subjects and human patients.

In alternative embodiments, formulation may be administered in a dosage or dosage form that delivers approximately 25 to 50 mg CBD per day, or approximately 30 to 45 mg CBD per day, or approximately 35 to 4 0mg CBD per day. Similarly, the dosage or dosage form may deliver approximately 1.5 to 3 mg THC per day, or approximately 2 to 2.5 mg THC per day. In a select embodiment, these dosages of CBD and THC may be delivered in combination with approximately 1500-3000 mg omega-3 fatty acids per day, for example in dosages or dosage forms taken once per day, twice per day or three times per day.

In accordance with the foregoing, aspects of the invention provide select cannabinoid and fatty acid formulations, for example comprising one or more cannabinoid-containing plant extracts in combination with one or more omega-3 fatty acid solvents. A pharmaceutically acceptable excipient may also be included. These formulations may be used for preventing or treating a variety of neurological disorders, including traumatic brain injury, concussion, post-concussive syndrome, neuroinflammation or sequelae thereof.

In select aspects of the present invention, the “major cannabinoid” is the predominant cannabinoid in the cannabinoid-containing plant extract. In select embodiments, the major cannabinoid will be CBD. In some embodiments, the major cannabinoid may for example be present at 12-14 mg/mL in solution with medium chain triglycerides (MCT) oil as a solvent. Medium chain triglycerides, in this context, are triglycerides comprised of aliphatic fatty acids in which 2 or 3 of the fatty acid chains are 6-12 carbons in length.

In some aspects of the present invention, the “minor cannabinoid” is the second most predominant cannabinoid, this may for example be THC, for example if the plant extract is from a Cannabis plant bred for high CBD content. In some embodiments, THC may for example be present at 0.5-1.0 mg/mL. The CBD and THC may for example be present in solution, for example in MCT oil.

The “other cannabinoids” herein are defined as all of the remaining cannabinoids that are present in the cannabinoid-containing plant extract other than THC or CBD.

The cannabinoid-containing components of the formulation, for example in the form of a plant extract, may be combined with one or more omega-3 fatty acid solvents, for example comprising of EPA and/or DHA. In addition, a pharmaceutically acceptable excipient may be included, and the formulation may be provided in a titratable dosage form.

Formulations may also include pharmaceutically acceptable terpenoids or terpenes, including plant-derived terpenoids or terpenes, such as astaxanthin or other sesquiterpenes, tetraterpenes, triterpenes, diterpenes or monoterpenes (see Thoppil and Bishayee, World J Hepatol. 2011 Sep. 27; 3 (9): 228-249).

A titratable dosage may for example be adapted to allow a patient to take the medication in doses smaller than the unit dose, wherein a “unit dose” is defined as the maximum dose of medication that can be taken at any one time or within a specific dosage period. Titration of doses will allow different patients to incrementally increase the dose until they feel that the medication is efficacious, as not all patients will require the same dose to achieve the same benefits. A person with a larger build or faster metabolism may require larger doses to achieve the same effect as another with a smaller build or slower metabolism. Therefore, a titratable dosage has advantages over a standard dosage form.

The term “traumatic brain injury” is defined herein as an injury that occurs to the brain as a result of biomechanical forces acting on the brain, whether directly or indirectly. Forces can still be transmitted to the brain, causing a traumatic brain injury, if the point of impact was on another part of the body and forces were transmitted indirectly to the brain. A traumatic brain injury can occur from acceleration/deceleration forces alone, even in the absence of an impact to body.

A traumatic brain injury can cause one or more of the following symptoms: headache, dizziness, nausea/vomiting, fatigue, phonophobia, photophobia, difficulty reading, memory difficulties, difficulty reading, speech difficulties, cognitive difficulties, slower thinking, brain fog, blurry vision, double vision, balance impairment, mood changes, irritability, sadness, anxiety, sleep disturbances, insomnia, and less commonly, nightmares, anosmia, and seizures.

A concussion is a traumatic brain injury. A concussion does not a require loss of consciousness, vomiting, post-traumatic amnesia, or positive findings on routine neuroimaging modalities such as computed tomography (CT) or magnetic resonance imaging (MRI) as a diagnostic requirement.

Post-concussive syndrome is defined herein as persistent symptoms following a traumatic brain injury lasting far beyond the typical recovery period. In the case of a concussion, the typical recovery period is within 7-14 days. Post-concussive syndrome resulting from a concussion is defined herein as persistent symptoms lasting longer than 3 months.

In select embodiments, formulations may be adapted to be delivered in such a way as to target one or more of the following: sublingual, buccal, oral, rectal, nasal, parenteral and via the pulmonary system. Formulations may for example be in one or more of the following forms: gel, gel spray, tablet, liquid, capsule, by injection, or for vaporization.

Conventional pharmaceutical practice may be employed to provide suitable formulations or compositions to administer the formulations to subjects. Routes of administration may for example include, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, sublingual or oral administration. Therapeutic formulations may be in the form of liquid solutions or suspensions; for oral administration, formulations may be in the form of tablets or capsules; for intranasal formulations, in the form of powders, nasal drops, or aerosols; and for sublingual formulations, in the form of drops, aerosols or tablets.

Methods well known in the art for making formulations are found in, for example, “Remington's Pharmaceutical Sciences” (20th edition), ed. A. Gennaro, 2000, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Pharmaceutical compositions of the present invention may be in any form which allows for the composition to be administered to a patient. For example, the composition may be in the form of a solid, liquid or gas (aerosol). Pharmaceutical composition of the invention are formulated so as to allow the active ingredients contained therein to be bioavailable upon administration of the composition to a patient. Compositions that will be administered to a patient may take the form of one or more dosage units, where for example, a tablet, capsule or cachet may be a single dosage unit, and a container of the compound in aerosol form may hold a plurality of dosage units.

Materials used in preparing the pharmaceutical compositions should be pharmaceutically pure and non-toxic in the amounts used. The inventive compositions may include one or more compounds (active ingredients) known for a particularly desirable effect. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes a formulation of the present invention as described herein, in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid formulation for oral administration, the composition may be formulated into a powder, granule, compressed tablet, pill, capsule, cachet, chewing gum, wafer, lozenges, or the like form. Such a solid composition will typically contain one or more inert diluents or edible carriers. In addition, one or more of the following adjuvants may be present: binders such as syrups, acacia, sorbitol, polyvinylpyrrolidone, carboxymethylcellulose, ethyl cellulose, microcrystalline cellulose, gum tragacanth or gelatin, and mixtures thereof; excipients such as starch, lactose or dextrins, disintegrating agents such as alginic acid, sodium alginate, Primogel, corn starch and the like; lubricants such as magnesium stearate or Sterotex; fillers such as lactose, mannitols, starch, calcium phosphate, sorbitol, methylcellulose, and mixtures thereof; lubricants such as magnesium stearate, high molecular weight polymers such as polyethylene glycol, high molecular weight fatty acids such as stearic acid, silica, wetting agents such as sodium lauryl sulfate, glidants such as colloidal silicon dioxide; sweetening agents such as sucrose or saccharin, a flavoring agent such as peppermint, methyl salicylate or orange flavoring, and a coloring agent. When the composition is in the form of a capsule, e.g., a gelatin capsule, it may contain, in addition to materials of the above type, a liquid carrier such as polyethylene glycol or a fatty oil.

The formulation may be in the form of a liquid, e.g., an elixir, syrup, solution, aqueous or oily emulsion or suspension, or even dry powders which may be reconstituted with water and/or other liquid media prior to use. The liquid may be for oral administration or for delivery by injection, as two examples. When intended for oral administration, preferred compositions contain, in addition to the present compounds, one or more of a sweetening agent, thickening agent, preservative (e.g., alkyl p-hydoxybenzoate), dye/colorant and flavor enhancer (flavorant). In a composition intended to be administered by injection, one or more of a surfactant, preservative (e.g., alkyl p-hydroxybenzoate), wetting agent, dispersing agent, suspending agent (e.g., sorbitol, glucose, or other sugar syrups), buffer, stabilizer and isotonic agent may be included. The emulsifying agent may be selected from lecithin or sorbitol monooleate.

The liquid pharmaceutical formulations of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The pharmaceutical formulation may be intended for topical administration, in which case the carrier may suitably comprise a solution, emulsion, ointment, cream or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The formulation may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. Low-melting waxes are preferred for the preparation of a suppository, where mixtures of fatty acid glycerides and/or cocoa butter are suitable waxes. The waxes may be melted, and the aminocyclohexyl ether compound is dispersed homogeneously therein by stirring. The molten homogeneous mixture is then poured into convenient sized molds, allowed to cool and thereby solidify.

The formulation may include various materials which modify the physical form of a solid or liquid dosage unit. For example, the composition may include materials that form a coating shell around the active ingredients. The materials which form the coating shell are typically inert, and may be selected from, for example, sugar, shellac, and other enteric coating agents. Alternatively, the active ingredients may be encased in a gelatin capsule or cachet.

The pharmaceutical formulation may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system which dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit.

Some biologically active compounds may be in the form of the free base or in the form of a pharmaceutically acceptable salt such as the hydrochloride, sulfate, phosphate, citrate, fumarate, methanesulfonate, acetate, tartrate, maleate, lactate, mandelate, salicylate, succinate and other salts known in the art. The appropriate salt would be chosen to enhance bioavailability or stability of the compound for the appropriate mode of employment (e.g., oral or parenteral routes of administration).

The present invention also provides kits that contain a pharmaceutical formulation, together with instructions for the use of the formulation. Preferably, a commercial package will contain one or more unit doses of the formulation.

Formulations which are light and/or air sensitive may require special packaging and/or formulation. For example, packaging may be used which is opaque to light, and/or sealed from contact with ambient air, and/or formulated with suitable coatings or excipients.

The formulations of the invention can be provided alone or in combination with other compounds (for example, small molecules, nucleic acid molecules, peptides, or peptide analogues), in the presence of a carrier or any pharmaceutically or biologically acceptable carrier. As used herein “pharmaceutically acceptable carrier” or “excipient” includes any and all solvents, dispersion media, coatings, antibacterial and antifungal agents, isotonic and absorption delaying agents, and the like that are physiologically compatible. The carrier can be suitable for any appropriate form of administration. Pharmaceutically acceptable carriers generally include sterile aqueous solutions or dispersions and sterile powders. Supplementary active compounds can also be incorporated into the formulations.

An “effective amount” of a formulation according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a formulation may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the formulation or active compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. For any particular subject, the timing and dose of treatments may be adjusted over time (e.g., timing may be daily, every other day, weekly, monthly) according to the individual need and the professional judgment of the person administering or supervising the administration of the compositions.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing.

Citation of references herein is not an admission that such references are prior art to the present invention. Any priority document(s) and all publications, including but not limited to patents and patent applications, cited in this specification are incorporated herein by reference. All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual publication were specifically and individually indicated to be incorporated by reference herein and as though fully set forth herein. The invention includes all embodiments and variations substantially as hereinbefore described and with reference to the examples and drawings. In some embodiments, the invention excludes steps that involve medical or surgical treatment.

The results of the clinical studies described in the following Examples provide evidence that a drug consisting, in part, of a cannabinoid-containing plant extract is surprisingly effective in the treatment of concussion, traumatic brain injury, and post-concussive syndrome.

### EXAMPLES

### Example 1: Human Pre- vs. Post-Treatment Study

This Example compares the SCAT3 symptom scores of 32 patient-subjects (16 male, 16 female) with PCS greater than 3 months in duration before and after treatment with a cannabinoid containing plant extract from a CBD dominant strain of Cannabis sativa in solution with MCT oil. The subjects were taken from a convenience sample of patients seeing a neurosurgeon for management of post-concussive syndrome. The ratio of THC:CBD was approximately 16:1. The CBD content was approximately 12.8 mg/mL and the THC content was approximately 0.8 mg/mL. Patients were instructed to take 1.5 mL of the Cannabis oil twice per day, once in the morning and once in the early afternoon. Patient were accordingly treated so as to provide a daily dosage of CBD of approximately 38.4 mg, and a daily dosage of THC of approximately 2.4 mg. These patients were also treated with 1-3 g DHA per day.

Prior to starting the treatment protocol, subjects were counseled in-person at the study site on the proper dosage, storage and use of the Cannabis oil and a pre-treatment SCAT3 symptom questionnaire was administered. 7-10 days after initiation of the treatment protocol, patient-subjects were contacted via phone by clinical research staff and a post-treatment SCAT3 symptom questionnaire was administered. Pre- and post-treatment symptom score totals were compared using a paired, two-tailed Student's t-test.

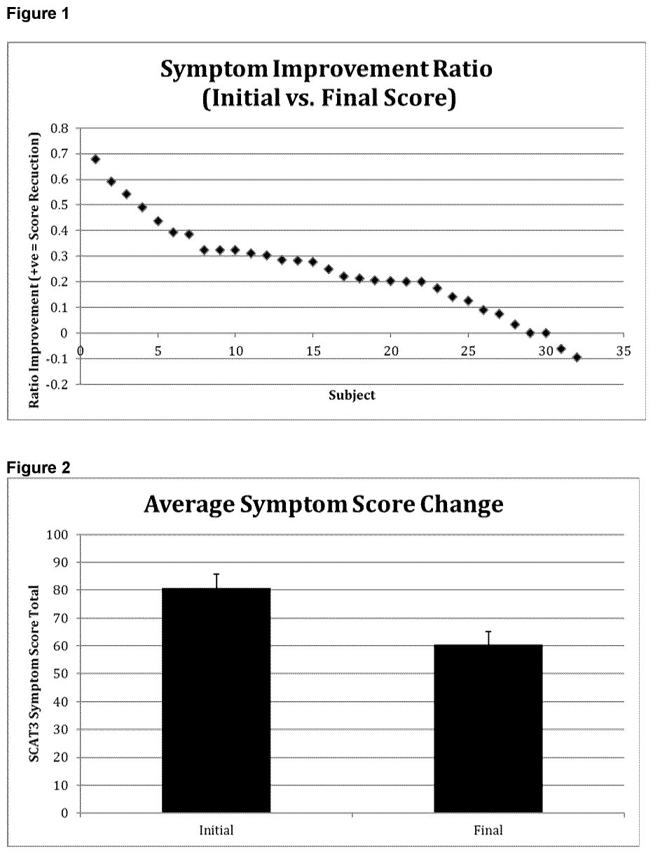

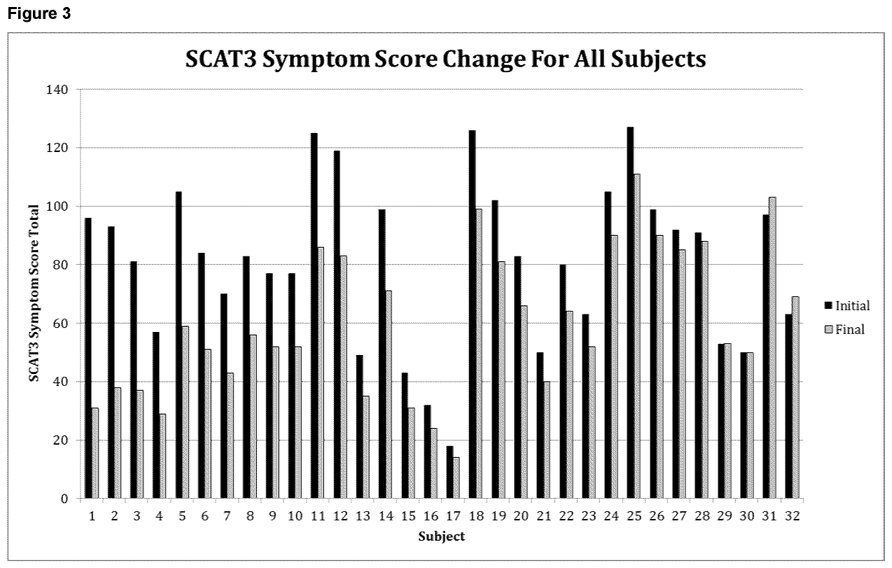

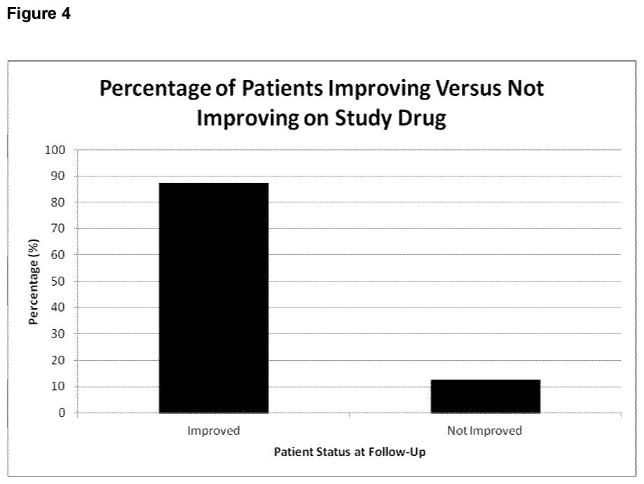

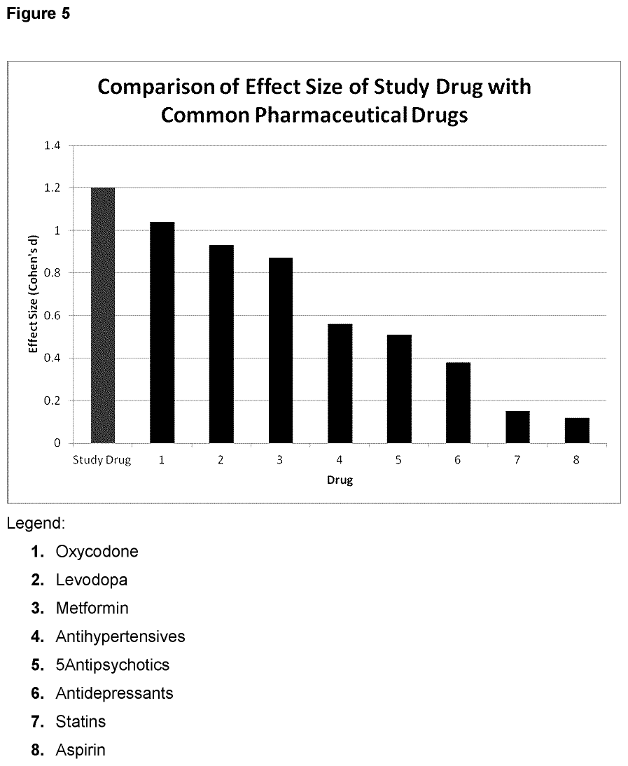

Pre-treatment scores ranged from 18 to 127 with an average of 80.91 and a median score of 83. Post-treatment scores ranged from 14 to 111 with an average of 60.41 and a median score of 54.5. The average ratio change pre- versus post-treatment is a 24.81% reduction of symptom score (FIG. 1). The average reduction in symptom score is 20.5 points (FIG. 2). The paired, two-tailed Student's t-test demonstrated significant differences between symptom scores pre- and post-treatment, p<0.001. The effect size of the sample using Cohen's d is 1.20 (FIG. 3). Based on Sawilowsky (2009), “New effect size rules of thumb” in the Journal of Modern Applied Statistical Methods, an effect size of 1.20 would be considered as “very large”. There were no reports of psychoactive adverse effects. One subject reported increased tinnitus as a non-serious adverse effect and subsequently discontinued use of the Cannabis oil prior to follow-up, and their data was excluded.

### Example 2: Case Reports from Example 1

Subject 6 is a 46-year-old right-handed male who sustained a concussion following a motor vehicle accident over two years ago and had progressed into a post-concussive syndrome. He experienced the entire constellation of post-concussive symptoms (physical, cognitive, emotional, and sleep-related symptoms) and was struggling with significant anxiety and speech difficulties following the injury. He had difficulty carrying out a normal conversation and would constantly stutter his speech. He had significant difficulty being in a social environment due to his anxiety and avoided social interactions when possible. He also took a significant amount of prescription medication to control his symptoms, including Percocet and Trazodone. His initial SCAT3 symptom score was 84. After taking the Cannabis oil formulation described in Example 1, his symptom score decreased to 51. His wife noticed almost immediate improvement in all areas, in particular his speech and anxiety. He was able to carry on a conversation with essentially no speech impediment. He could tolerate being in social environments for longer durations (hours). He was able to significantly decrease his reliance on Percocet and Trazodone to ameliorate his symptoms. The subject reported that when he had to fly out of the country on a trip and could not take the Cannabis oil for a week, his symptoms returned. When he started taking the Cannabis oil again, the symptoms improved again.

Subject A.B. is a 57-year-old left-handed female who sustained a concussion about two years ago following a physical assault when she was pushed into a wall. She did lose consciousness and experienced some post-traumatic amnesia at the time. She had progressed into a post-concussive syndrome with persistent symptoms. Originally, she was part of the study described in Example 1, but due to exceptional circumstances, her data was excluded from the analysis. Her initial symptom score was 71. After taking the Cannabis oil described in Example 1, her symptom score actually increased to 86. Final symptom scores were obtained 7-10 days following the first dose of Cannabis oil taken by the subject. The subject reported that she did not feel that the medication was showing any positive effect. She was instructed to continue taking the medication, in case there was some delay in the response. In subsequent follow-up with the neurosurgeon approximately 6 weeks after taking the first dose, she reported that she was feeling improvement in her symptoms. Data was not collected for Subject A.B. at 6 weeks. However, this case shows that some patients may not respond to the formulations immediately, and may for example take as long as 6 weeks to respond.

### Example 3: Clinical Trial

A Phase 2 a pilot study was designed as a retrospective case series with subjects (n=100) receiving the formulation of Example 1, and qualifying for the study based on specific inclusion and exclusion criteria. The study population was made up of patients who were evaluated to have refractory post-concussion syndrome (PCS), defined by persisting complaints associated with PCS. Subjects were monitored prior to receiving and following treatment utilizing validated scales. The formulation used to treat the patients was (as in Example 1): 0.8 mg/mL THC, 12.8 mg/mL CBD (in Cannabis oil, given in a 1.5 mL dose twice per day) and 1-3 g DHA as delivery oil each day.

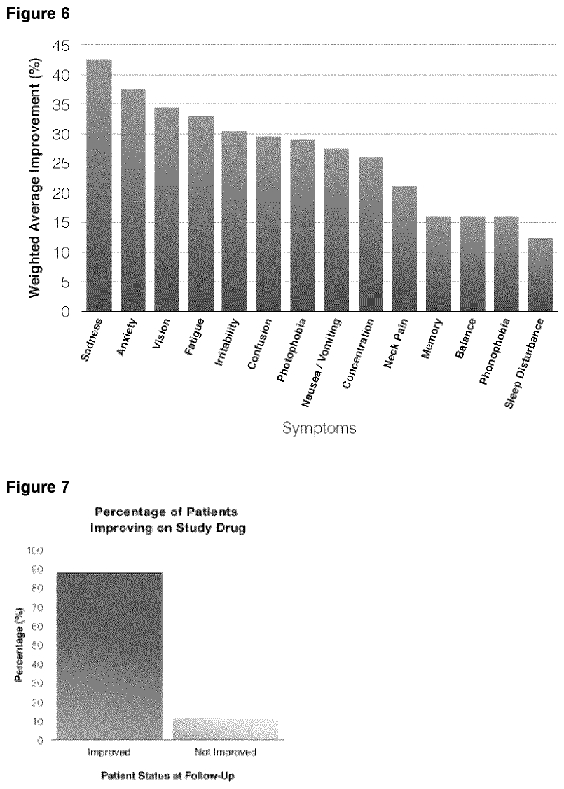

Patients in this study demonstrated clinically significant improvements in cognitive function, speech, headaches and additional debilitating symptoms. Results are illustrated in FIGS. 6 and 7, reflecting the percentage improvement in symptoms of 100 concussed patients on the formulation based on SCAT-3 (validated concussion scale). The resulting p-value (p<0.001) and treatment effect size of 1.32 are robust values, indicating both clinical and statistical significance.

### REFERENCES

Bailes, J. E. and V. Patel (2014). “The potential for DHA to mitigate mild traumatic brain injury.” Mil Med 179 (11 Suppl): 112-116.

Barrett, E. C., M. I. McBurney and E. D. Ciappio (2014). “omega-3 fatty acid supplementation as a potential therapeutic aid for the recovery from mild traumatic brain injury/concussion.” Adv Nutr 5 (3): 268-277.

Blaylock, R. L. and J. Maroon (2011). “Immunoexcitotoxicity as a central mechanism in chronic traumatic encephalopathy-A unifying hypothesis.” Surg Neurol Int 2: 107.

Broglio, S. P., J. T. Eckner, H. L. Paulson and J. S. Kutcher (2012).

“Cognitive decline and aging: the role of concussive and subconcussive impacts.” Exerc Sport Sci Rev 40 (3): 138-144.

Chagas, M. H. N., J. A. S. Crippa, A. W. Zuardi, J. E. C. Hallak, J. P. Machado-de-Sousa, C. Hirotsu, L. Maia, S. Tufik and M. L. Andersen (2013). “Effects of acute systemic administration of cannabidiol on sleep-wake cycle in rats.” Journal of Psychopharmacology 27 (3): 312-316.

Desai, A., K. Kevala and H. Y. Kim (2014). “Depletion of brain docosahexaenoic acid impairs recovery from traumatic brain injury.” PLoS One 9 (1): e86472.

Ellulu, M. S., H. Khaza'ai, Y. Abed, A. Rahmat, P. Ismail and Y. Ranneh (2015). “Role of fish oil in human health and possible mechanism to reduce the inflammation.” Inflammopharmacology 23 (2-3): 79-89.

Fowler, C. J. (2003). “Plant-derived, synthetic and endogenous cannabinoids as neuroprotective agents. Non-psychoactive cannabinoids, ‘entourage’ compounds and inhibitors of N-acyl ethanolamine breakdown as therapeutic strategies to avoid pyschotropic effects.” Brain Res Brain Res Rev 41 (1): 26-43.

Giza, C. C. and D. A. Hovda (2014). “The new neurometabolic cascade of concussion.” Neurosurgery 75 Suppl 4: S24-33.

Jain, A. P., K. K. Aggarwal and P. Y. Zhang (2015). “Omega-3 fatty acids and cardiovascular disease.” Eur Rev Med Pharmacol Sci 19 (3): 441-445.

Katz, P. S., J. K. Sulzer, R. A. Impastato, S. X. Teng, E. K. Rogers and P. E. Molina (2015). “Endocannabinoid degradation inhibition improves neurobehavioral function, blood-brain barrier integrity, and neuroinflammation following mild traumatic brain injury.” J Neurotrauma 32 (5): 297-306.

Langlois, J. A., W. Rutland-Brown and M. M. Wald (2006). “The epidemiology and impact of traumatic brain injury: a brief overview.” J Head Trauma Rehabil 21 (5): 375-378.

Morse, N. L. (2012). “Benefits of docosahexaenoic acid, folic acid, vitamin D and iodine on foetal and infant brain development and function following maternal supplementation during pregnancy and lactation.” Nutrients 4 (7): 799-840.

Patterson, Z. R. and M. R. Holahan (2012). “Understanding the neuroinflammatory response following concussion to develop treatment strategies.” Front Cell Neurosci 6: 58.

Rocchetti, M., A. Crescini, S. Borgwardt, E. Caverzasi, P. Politi, Z. Atakan and P. Fusar-Poli (2013). “Is Cannabis neurotoxic for the healthy brain? A meta-analytical review of structural brain alterations in non-psychotic users.” Psychiatry Clin Neurosci 67 (7): 483-492.

Sarne, Y., F. Asaf, M. Fishbein, M. Gafni and O. Keren (2011). “The dual neuroprotective-neurotoxic profile of cannabinoid drugs.” Br J Pharmacol 163 (7): 1391-1401.

Sanchez-Blazquez, P., M. Rodriguez-Munoz and J. Garzon (2014). “The cannabinoid receptor 1 associates with NMDA receptors to produce glutamatergic hypofunction: implications in psychosis and schizophrenia.” Front Pharmacol 4: 169.

Shultz, S. R., D. F. MacFabe, K. A. Foley, R. Taylor and D. P. Cain (2012). “Sub-concussive brain injury in the Long-Evans rat induces acute neuroinflammation in the absence of behavioral impairments.” Behav Brain Res 229 (1): 145-152.

Svizenska, I., P. Dubovy and A. Sulcova (2008). “Cannabinoid receptors 1 and 2 (CB1 and CB2), their distribution, ligands and functional involvement in nervous system structures—a short review.” Pharmacol Biochem Behav 90 (4): 501-511.

Vendel, E. and E. C. de Lange (2014). “Functions of the CB1 and CB 2 receptors in neuroprotection at the level of the blood-brain barrier.” Neuromolecular Med 16 (3): 620-642.

Woodhams, S. G., D. R. Sagar, J. J. Burston and V. Chapman (2015). “The role of the endocannabinoid system in pain.” Handb Exp Pharmacol 227: 119-143.

1. A pharmaceutical formulation, comprising:
cannabidiol (CBD);
tetrahydrocannabinol (THC), where the weight ratio of CBD:THC is equal to or between 10:1 and 20:1; and,
one or more omega-3 fatty acids, comprising eicosahexaenoic acid (EPA) and/or docosahexaenoic acid (DHA) or pharmaceutically acceptable salts thereof, providing a combined concentration of EPA and/or DHA of 200-3000 mg/mL.

2. The pharmaceutical formulation of claim 1, wherein the weight ratio of CBD:THC is between 14:1 and 18:1.

3. The pharmaceutical formulation of claim 1 or 2, further comprising a pharmaceutically acceptable excipient.

4. The formulation of any one of claims 1 to 3, wherein CBD is present at 10-15 mg/ml.

5. The formulation of any one of claims 1 to 3, wherein CBD is present at 12-14 mg/ml.

6. The formulation of any one of claims 1 to 3, wherein CBD is present at approximately 12.8 mg/ml.

7. The formulation of any one of claims 1 to 6, wherein THC is present at 0.5 to 1 mg/ml.

8. The formulation of any one of claims 1 to 6, wherein THC is present at 0.6-0.9 mg/ml.

9. The formulation of any one of claims 1 to 6, wherein THC is present at approximately 0.8 mg/ml.

10. The formulation of any one of claims 1 to 9, wherein the weight ratio of CBD:THC is approximately 16:1

11. The formulation of any one of claims 1 to 10, wherein the CBD and THC are from a plant extract.

12. The formulation of claim 11, wherein the plant is a Cannabis sativa or Cannabis indica plant.

13. The formulation of any one of claims 1 to 12, wherein the CBD and THC are dissolved in the formulation.

14. The formulation of any one of claims 1 to 13, further comprising one or more of: cannabigerol (CBG), cannabichromene (CBC), tetrahydrocannabivirin (THCV), cannabidivirin (CBDV), cannabidiolic acid (CBDA) or astaxanthin.

15. The formulation of any one of claims 1 to 14, further comprising a non-cannabinoid-containing fraction.

16. The formulation of claim 15, wherein the non-cannabinoid-containing fraction comprises one or more of: terpenes, terpenoids, sterols, triglycerides, alkanes, squalene, tocopherol, carotenoids, chlorophyll, flavonoid glycosides, or alkaloids.

17. The formulation of claim 15 or 16, wherein the non-cannabinoid-containing fraction makes up between 1% and 45% by weight of the formulation.

18. Use of the formulation of any one of claims 1 to 17, to prophylactically or therapeutically treat traumatic brain injury, concussion, post-concussive syndrome, neuroinflammation or sequelae thereof, wherein the formulation is optionally for use in an amount of from 0.25 mL to 5 mL per day.

19. Use of the formulation of any one of claims 1 to 17, to formulate a medicament for prophylactically or therapeutically treating traumatic brain injury, concussion, post-concussive syndrome, neuroinflammation or sequelae thereof, wherein the formulation is optionally for use in an amount of from 0.25 mL to 5 mL per day.

20. A method of prophylactically or therapeutically treating a nervous system disorder in a subject in need thereof, comprising administering to the subject an effective amount of a formulation comprising cannabidiol (CBD) and tetrahydrocannabinol (THC), where the weight ratio of CBD:THC is equal to or between 10:1 and 20:1, wherein the disorder is traumatic brain injury, concussion, post-concussive syndrome, neuroinflammation or sequelae thereof.

21. The method of claim 20, wherein the formulation further comprises one or more omega-3 fatty acids, comprising eicosahexaenoic acid (EPA) and/or docosahexaenoic acid (DHA) or pharmaceutically acceptable salts thereof, providing a combined concentration of EPA/DHA of 200-3000 mg/mL.

22. The method of claim 20, further comprising treating the patient with an effective amount of one or more omega-3 fatty acids, comprising eicosahexaenoic acid (EPA) and/or docosahexaenoic acid (DHA) or pharmaceutically acceptable salts thereof.

23. The method of claim 21 or 22, wherein EPA and/or DHA are administered in an effective amount of 0.5 to 5 g/day.

24. The method of any one of claims 20 to 23, wherein the weight ratio of CBD:THC is between 14:1 and 18:1.

25. The method of any one of claims 20 to 24, wherein the formulation further comprises a pharmaceutically acceptable excipient.

26. The method of any one of claims 20 to 25, wherein CBD is present in the formulation at 10-15 mg/ml.

27. The method of any one of claims 20 to 25, wherein CBD is present in the formulation at 12-14 mg/ml.

28. The method of any one of claims 20 to 25, wherein CBD is present in the formulation at approximately 12.8 mg/ml.

29. The method of any one of claims 20 to 28, wherein THC is present in the formulation at 0.5 to 1 mg/ml.

30. The method of any one of claims 20 to 28, wherein THC is present in the formulation at 0.6-0.9 mg/ml.

31. The method of any one of claims 20 to 28, wherein THC is present in the formulation at approximately 0.8 mg/ml.

32. The method of any one of claims 20 to 31, wherein the weight ratio of CBD:THC in the formulation is approximately 16:1

33. The method of any one of claims 20 to 32, wherein the CBD and THC in the formulation are from a plant extract.

34. The method of claim 33, wherein the plant is a Cannabis sativa or Cannabis indica plant.

35. The method of any one of claims 20 to 34, wherein the CBD and THC are dissolved in the formulation.

36. The method of any one of claims 20 to 35, wherein the formulation further comprises one or more of: cannabigerol (CBG), cannabichromene (CBC), tetrahydrocannabivirin (THCV), cannabidivirin (CBDV), cannabidiolic acid (CBDA) or astaxanthin.

37. The method of any one of claims 20 to 36, wherein the formulation further comprises a non-cannabinoid-containing fraction.

38. The method of claim 37, wherein the non-cannabinoid-containing fraction comprises one or more of: terpenes, terpenoids, sterols, triglycerides, alkanes, squalene, tocopherol, carotenoids, chlorophyll, flavonoid glycosides, or alkaloids.

39. The method of claim 37 or 38, wherein the non-cannabinoid-containing fraction makes up between 1% and 45% by weight of the formulation.

40. The method of any one of claims 20 to 39, wherein the subject is mammalian.

41. The method of claim 40, wherein the subject is a human patient.

42. The method according to claim 41, wherein the formulation is administered in dosage that delivers approximately 25 to 50 mg CBD per day.

43. The method according to claim 41, wherein the formulation is administered in dosage that delivers approximately 30 to 45 mg CBD per day.

44. The method according to claim 41, wherein the formulation is administered in dosage that delivers approximately 35 to 40 mg CBD per day.

45. The method according to any one of claims 41 to 44, wherein the formulation is administered in dosage that delivers approximately 1.5 to 3 mg THC per day.

46. The method according to any one of claims 41 to 44, wherein the formulation is administered in dosage that delivers approximately 2 to 2.5 mg THC per day.

47. The method according to any one of claims 41 to 46, wherein the formulation is administered in a dosage that delivers a combined amount of EPA/DHA of approximately 1500-3000 mg per day.

48. The method of any one of claims 41 to 47, wherein the formulation is delivered in a dosage form taken once per day, twice per day or three times per day.

49. The formulation of any one of claims 1 to 17, for use to prophylactically or therapeutically treat traumatic brain injury, concussion, post-concussive syndrome, neuroinflammation or sequelae thereof.

50. The formulation of any one of claims 1 to 17, for use to formulate a medicament for prophylactically or therapeutically treating traumatic brain injury, concussion, post-concussive syndrome, neuroinflammation or sequelae thereof.

51. A formulation for use in the prophylactic or therapeutic treatment of a nervous system disorder in a subject in need thereof, wherein the formulation comprises an effective amount of cannabidiol (CBD) and tetrahydrocannabinol (THC), where the weight ratio of CBD:THC is equal to or between 10:1 and 20:1, wherein the disorder is traumatic brain injury, concussion, post-concussive syndrome, neuroinflammation or sequelae thereof.

52. The formulation for use according to claim 51, wherein the formulation further comprises one or more omega-3 fatty acids, comprising eicosahexaenoic acid (EPA) and/or docosahexaenoic acid (DHA) or pharmaceutically acceptable salts thereof, providing a combined concentration of EPA/DHA of 200-3000 mg/mL.

53. The formulation for use according to claim 51, further comprising treating the patient with an effective amount of one or more omega-3 fatty acids, comprising eicosahexaenoic acid (EPA) and/or docosahexaenoic acid (DHA) or pharmaceutically acceptable salts thereof.

54. The formulation for use according to claim 52 or 53, wherein EPA and/or DHA are administered in an effective amount of 0.5 to 5 g/day.

55. The formulation for use according to any one of claims 51 to 54, wherein the weight ratio of CBD:THC is between 14:1 and 18:1.

56. The formulation for use according to any one of claims 51 to 55, wherein the formulation further comprises a pharmaceutically acceptable excipient.

57. The formulation for use according to any one of claims 51 to 56, wherein CBD is present in the formulation at 10-15 mg/ml.

58. The formulation for use according to any one of claims 51 to 56, wherein CBD is present in the formulation at 12-14 mg/ml.

59. The formulation for use according to any one of claims 51 to 56, wherein CBD is present in the formulation at approximately 12.8 mg/ml.

60. The formulation for use according to any one of claims 51 to 59, wherein THC is present in the formulation at 0.5 to 1 mg/ml.

61. The formulation for use according to any one of claims 51 to 59, wherein THC is present in the formulation at 0.6-0.9 mg/ml.

62. The formulation for use according to any one of claims 51 to 59, wherein THC is present in the formulation at approximately 0.8 mg/ml.

63. The formulation for use according to any one of claims 51 to 62, wherein the weight ratio of CBD:THC in the formulation is approximately 16:1

64. The formulation for use according to any one of claims 51 to 63, wherein the CBD and THC in the formulation are from a plant extract.

65. The formulation for use according to claim 64, wherein the plant is a Cannabis sativa or Cannabis indica plant.

66. The formulation for use according to any one of claims 51 to 65, wherein the CBD and THC are dissolved in the formulation.

67. The formulation for use according to any one of claims 51 to 66, wherein the formulation further comprises one or more of: cannabigerol (CBG), cannabichromene (CBC), tetrahydrocannabivirin (THCV), cannabidivirin (CBDV), cannabidiolic acid (CBDA) or astaxanthin.

68. The formulation for use according to any one of claims 51 to 67, wherein the formulation further comprises a non-cannabinoid-containing fraction.

69. The formulation for use according to claim 68, wherein the non-cannabinoid-containing fraction comprises one or more of: terpenes, terpenoids, sterols, triglycerides, alkanes, squalene, tocopherol, carotenoids, chlorophyll, flavonoid glycosides, or alkaloids.

70. The formulation for use according to claim 68 or 69, wherein the non-cannabinoid-containing fraction makes up between 1% and 45% by weight of the formulation.

71. The formulation for use according to any one of claims 51 to 70, wherein the subject is mammalian.

72. The formulation for use according to claim 71, wherein the subject is a human patient.

73. The formulation for use according to claim 72, wherein the formulation is administered in dosage that delivers approximately 25 to 50 mg CBD per day.

74. The formulation for use according to claim 72, wherein the formulation is administered in dosage that delivers approximately 30 to 45 mg CBD per day.

75. The formulation for use according to claim 72, wherein the formulation is administered in dosage that delivers approximately 35 to 40 mg CBD per day.

76. The formulation for use according to any one of claims 72 to 75, wherein the formulation is administered in dosage that delivers approximately 1.5 to 3 mg THC per day.

77. The formulation for use according to any one of claims 72 to 75, wherein the formulation is administered in dosage that delivers approximately 2 to 2.5 mg THC per day.

78. The formulation for use according to any one of claims 72 to 77, wherein the formulation is administered in a dosage that delivers a combined amount of EPA/DHA of approximately 1500-3000 mg per day.

79. The formulation for use according to any one of claims 72 to 78, wherein the formulation is delivered in a dosage form taken once per day, twice per day or three times per day.
